SEQUENCE LISTING (14 sequences)

SEQ ID NO: 1 - protein, 13 aa, Glycine max
TSLDFPALSWLRL

SEQ ID NO: 2 - protein, 12 aa, Glycine max
SLDFPALSWLRL

SEQ ID NO: 3 - protein, 11 aa, Glycine max
LDFPALSWLRL

SEQ ID NO: 4 - protein, 10 aa, Glycine max
DFPALSWLRL

SEQ ID NO: 5 - protein, 9 aa, Glycine max
FPALSWLRL

SEQ ID NO: 6 - protein, 8 aa, Glycine max
PALSWLRL

SEQ ID NO: 7 - protein, 7 aa, Glycine max
ALSWLRL

SEQ ID NO: 8 - protein, 12 aa, Glycine max
TSLDFPALSWLR

SEQ ID NO: 9 - protein, 11 aa, Glycine max
TSLDFPALSWL

SEQ ID NO: 10 - protein, 10 aa, Glycine max
TSLDFPALSW

SEQ ID NO: 11 - protein, 9 aa, Glycine max
TSLDFPALS

SEQ ID NO: 12 - protein, 8 aa, Glycine max
TSLDFPAL

SEQ ID NO: 13 - protein, 7 aa, Glycine max
TSLDFPA

SEQ ID NO: 14 - DNA, 39 bp, Glycine max
acttctttagattttcctgctctttcttggttaagatta

(19) United States
(12) Patent Application Publication
(10) Pub. No.: US 2016/0002294 A1
(43) Pub. Date: Jan. 7, 2016

(54) NOVEL POLYPEPTIDES HAVING SATIETY HORMONE RELEASING ACTIVITY

(71) Applicant: SOLAE LLC, St. Louis, MO (US)

(72) Inventors: Barry M. Tulk, St. Louis, MO (US); Elaine S. Krul, St. Louis, MO (US); Nancy J. McGraw, St. Louis, MO (US)

(73) Assignee: Solae, LLC, St. Louis, MO (US)

(21) Appl. No.: 14/764,038

(22) PCT Filed: Jan. 28, 2014
(86) PCT No.: PCT/US2014/013362

§ 371 Date: Jul. 28, 2015

Related U.S. Application Data
(60) Provisional application No. 61/757,556, filed on Jan. 28, 2013.

(51) Int. Cl. C07K 7/08; A23L 1/305

(52) U.S. Cl. CPC C07K 7/08; A23L 1/3053; A61K 38/00

(57) ABSTRACT

The present invention relates to novel polypeptides having satiety hormone releasing activity (e.g., cholecystokinin (CCK) and/or glucagon-like peptide-1 (GLP-1) releasing activity).

### CROSS REFERENCE TO RELATED APPLICATION

This application claims the priority of U.S. provisional application No. 61/757,556 filed Jan. 28, 2013, which is hereby incorporated by reference in its entirety.

### FIELD OF THE INVENTION

The present invention generally relates to novel polypeptides and uses thereof. In particular, the novel polypeptides of the invention have satiety hormone releasing activity (e.g., cholecystokinin (CCK) and/or glucagon-like peptide-1 (GLP-1) releasing activity).

### BACKGROUND OF THE INVENTION

The number and rate of overweight and obese individuals and the diseases associated with increased body weight are rising in the United States and throughout the World. While there is no single underlying cause, a contributing factor may be the sedentary lifestyles of many individuals and the concomitant consumption of higher calorie foods, including “fast food.” Most “fast food” tends to be high in fat and/or sugar.

One viable target for combating the increased body weight epidemic may be induction of CCK. CCK is a peptide hormone released into the circulation by gastrointestinal cells in response to nutrients, specifically protein or lipids consumed as a meal. CCK acts as a neurotransmitter, and neuromodulator in the central and the peripheral nervous systems. CCK is released from I enteroendocrine cells of the duodenum and jejunum in response to nutrients (e.g., protein and fat) that enter the gastrointestinal lumen after a meal. Once released, CCK initiates a number of responses coordinated to promote digestion and regulate food intake, including mediating bile emptying from the gall bladder, regulating the release of digestive enzymes from the pancreas, controlling gastric emptying by regulation of the pyloric sphincter, as well as neuronal signaling to the central nervous system by vagal afferent neurons. Neuronal CCK is believed to mediate a number of events within the central nervous system, including modulating dopaminergic neurotransmission and anxiogenic effects, as well as affecting cognition and nociception (J. N. Crawley and R. L. Corwin, 1994, Peptides, 15:731-755; N. S. Baber, C. T. Dourish, and D. R. Hill, Pain (1989), 39(3), 307-28; and P. De Tullio, J. Delarge and B. Pirotte, Expert Opinion on Investigational Drugs (2000), 9(1), 129-146). CCK has been shown to mediate its diverse hormonal and neuromodulatory functions through two receptor subtypes: the CCK-A (CCK-1) and CCK-B (CCK-2) subtypes (G. N. Woodruff and J. Hughes, Annu. Rev. Pharmacol. Toxicol. (1991), 31: 469-501). Both CCK-1 and CCK-2 receptor subtypes belong to the seven transmembrane G-protein-coupled superfamily of receptors. A number of studies suggest that CCK mediates its satiety effect through the CCK-1 receptor, which is believed to relay the postprandial satiety signal by the vagal afferents to the central nervous system inducing a “feeling” of satiety (G. P. Smith et al., Science 213 (1981) pp. 1036-1037; and J. N. Crawley et al., J. Pharmacol. Exp. Ther., 257 (1991) pp. 1076-1080).

CCK has been shown to exert some direct actions that induce satiety, including the inhibition of gastric emptying, inhibition of gastric acid secretion, and stimulation of gallbladder contraction. Whether through these direct effects on gastric emptying and intestinal digestion or through central nervous system pathways, CCK induces a sense of satiety which typically results in the consumption of fewer calories.

Another viable target for combating the increased body weight epidemic may be induction of GLP-1. GLP-1 has been described as an incretin hormone with a large array of effects. GLP-1 was discovered in 1984 and found to be an important incretin (Nauck, M. A.; Kleine, N.; Orskov, C.; Hoist, J. J.; Willms, B.; Creutzfeldt, W., Diabetologia 1993, 36, 741-744). GLP-1 is released by L cells in the distal ileum in response to glucose and fatty acids, however, it is known that peptides directly induce and/or modulate GLP-1 release (Hira T et al. (2009) Am J Physiol Gastrointest Liver Physiol 297: G663-G671). GLP-1 is released into the circulation following a meal and potently stimulates the release of insulin from beta-cells in the pancreas in a glucose-dependent manner. Numerous additional effects have also been ascribed to GLP-1, including, stimulation of insulin biosynthesis, restoration of glucose sensitivity to the islets and stimulation of increased expression of the glucose transporter GLUT-2 and glucokinase. GLP-1 also has a number of effects on regulation of beta-cell mass, stimulation of replication and growth of existing beta-cells, inhibition of apoptosis and neogenesis of new beta-cells from duct precursor cells, which leads to reduced hepatic glucose output. Beneficial extra-pancreatic effects of GLP-1 have also been reported such as a direct effect on reducing hepatic lipid content and improving cardiac function (Abu-Hamdah R. et al. J Clin Endocrinol Metab. 2009 June; 94(6):1843-52. Epub 2009 Mar 31). In the gut, GLP-1 is a potent inhibitor of motility and gastric emptying and has also been shown to inhibit gastric acid secretion. The inhibition of gastric emptying leads to decreased food intake and reduced body weight over time (Flint, A.; Raben, A.; Astrup, A.; Hoist, J. J., J Clin Inv 1998, 101, 515-520; Zander, M.; Madsbad, S.; Madsen, J. L.; Hoist, J. J., Lancet 2002, 359, 824-830). GLP-1 has also been shown to have central effects on food intake through the action of GLP-1 receptors in the hypothalamic centers that control appetite (Maturitas. Barber T M et al. 2010 November; 67(3):197-202. doi: 10.1016/j.maturitas.2010.06.018. Epub 2010 Jul. 23).

In light of the increased body weight epidemic and the dearth of effective means of combating this, there is a need for a nutritious, readily accessible supplement, ingredient, and/or or food product that can be consumed and that promotes weight loss or control. To this end, the present invention is drawn to novel polypeptides having satiety hormone releasing activity (e.g., CCK and/or GLP-1 releasing activity).

### SUMMARY OF INVENTION

One aspect of the present invention encompasses the identification of novel polypeptides that have the ability to elicit CCK release from, for example, STC-1 cells, and polynucleotides encoding the same. In another aspect, the present invention encompasses the identification of novel polypeptides that have the ability to elicit GLP-1 release from, for example, STC-1 cells, and polynucleotides encoding the same. In a further aspect, the present invention encompasses the identification of novel polypeptides that have the ability to elicit CCK and GLP-1 release from, for example, STC-1 cells, and polynucleotides encoding the same.

In other aspects, the present invention encompasses a product comprising a polypeptide of the present invention (e.g., a food product comprising an edible material and a polypeptide described herein or a supplement comprising a polypeptide described herein).

In further other aspects, the present invention encompasses methods of eliciting CCK release from a cell, for example, an STC-1 cell, as a result of contacting said cell with a polypeptide of the present invention. In further aspects, the present invention encompasses methods of eliciting GLP-1 release from a cell, for example, an STC-1 cell, as a result of contacting said cell with a polypeptide of the present invention. In even further aspects, the present invention encompasses methods of eliciting CCK and GLP-1 release from a cell, for example, an STC-1 cell, as a result of contacting said cell with a polypeptide of the present invention. In specific aspects, the cell is an STC-1 cell.

In even further other aspects, the present invention encompasses methods of inducing satiety. In further aspects of methods of inducing satiety, a polypeptide of the invention is provided and elicits CCK release from a cell. In other further aspects of methods of inducing satiety, a polypeptide of the invention is provided and elicits GLP-1 release from a cell. In even other further aspects of methods of inducing satiety, a polypeptide of the invention is provided and elicits CCK and GLP-1 release from a cell.

### DESCRIPTION OF FIGURES

FIG. 1. CCK release dose-response curve for SEQ ID NO: 1. SEQ ID NO: 1 was synthesized using solid-phase methodology and used to stimulate release of CCK from STC-1 cells as described. Data was fit with a logistic function. From the regression data, EC50 was estimated to be 43 μM.

FIG. 2. GLP-1 release dose-response curve for SEQ ID NO: 1. SEQ ID NO: 1 was synthesized using solid-phase methodology and used to stimulate release of GLP-1 from STC-1 cells as described. Data was fit with a logistic function. From the regression data, EC50 was estimated to be 186 μM.

FIG. 3. CCK-release activity of truncated peptides. N- and C-terminal truncations of full-length SEQ ID NO: 1 peptide were synthesized using solid-phase methodology and used to stimulate CCK release from STC-1 cells as described. Data represent the results of three independent experiments, and is expressed as percent of CCK release activity stimulated by the full-length peptide.

FIG. 4. GLP-1-release activity of truncated peptides. N- and C-terminal truncations of full-length SEQ ID NO: 1 peptide were synthesized using solid-phase methodology and used to stimulate GLP-1 release from STC-1 cells as described. Data is expressed as percent of GLP-1 release activity stimulated by the full-length peptide.

### DETAILED DESCRIPTION OF THE INVENTION

CCK and GLP-1 promote satiety and slow gastric emptying, thereby imparting to a subject a feeling of being full. It has now been discovered that a novel polypeptide comprising an amino acid sequence of SEQ ID NO: 1, and variants thereof, has the ability to elicit CCK and GLP-1 release from a cell, for example, an STC-1 cell. The novel polypeptides of the present invention, and variants thereof, are more fully described as set forth herein.

Ingestion of food exerts a transient suppressive effect on appetite and further food intake. Among all properties of food, total energy content and the macronutrient composition (e.g., fat, carbohydrate, or protein) appear to be the major determinants of such satiety-related subjective feelings. A strong body of evidence indicates that the three macronutrients differ in extent to which they suppress hunger and energy intake. Proteins have been shown to be more satiating than fats, which in turn are more satiating than carbohydrates. Findings that some specific polypeptide structures derived from parent food proteins are biologically active have stimulated the search for peptides that may lead to the development of satiety inducing products (e.g., food products) targeted to control food intake and thus, affect body weight.

### I. NOVEL POLYPEPTIDES AND POLYNUCLEOTIDES ENCODING THE SAME

A specific aspect of the present invention relates to isolated polypeptides having a sequence identity of at least 50%, at least 55%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1. In another specific aspect, the polypeptides of the invention having a sequence identity of at least 50%, at least 55%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1, also have the ability to elicit CCK and/or GLP-1 release from, for example, STC-1 cells.

In other aspects, a polypeptide of the present invention comprises, consists essentially of, or consists of the amino acid sequence of SEQ ID NO: 1 or variants thereof and have the ability to elicit CCK and/or GLP-1 release from, for example, STC-1 cells. In the case of a polypeptide comprising a polypeptide of the present invention, these polypeptides can be of unlimited amino acid size or can be limited in size with an upper limit of about 1,000 amino acids, about 975 amino acids, about 950 amino acids about, about 925 amino acids, 900 amino acids, about 875 amino acids, about 850 amino acids, about 825 amino acids, about 800 amino acids, about 775 amino acids, about 750 amino acids, about 725 amino acids, about 700 amino acids, about 675 amino acids, about 650 amino acids, about 625 amino acids, about 600 amino acids, about 590 amino acids, about 580 amino acids, about 570 amino acids, about 560 amino acids, about 550 amino acids, about 540 amino acids, about 530 amino acids, about 520 amino acids, about 510 amino acids, about 500 amino acids, about 490 amino acids, about 480 amino acids, about 470 amino acids, about 460 amino acids, about 450 amino acids, about 440 amino acids, about 430 amino acids, about 420 amino acids, about 410 amino acids, about 400 amino acids, about 390 amino acids, about 380 amino acids, about 370 amino acids, about 360 amino acids, about 350 amino acids, about 340 amino acids, about 330 amino acids, about 320 amino acids, about 310 amino acids, about 300 amino acids, about 290 amino acids, about 280 amino acids, about 270 amino acids, about 260 amino acids, about 250 amino acids, about 240 amino acids, about 230 amino acids, about 220 amino acids, about 210 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 90 amino acids, about 80 amino acids, about 70 amino acids, about 60 amino acids, about 50 amino acids, about 40 amino acids, about 30 amino acids, about 20 amino acids, or about 15 amino acids.

With respect to polypeptides of the present invention as it relates to variants, these include, without limitation, insertions, substitutions, fragments, deletions, C-terminal truncations, and N-terminal truncations of SEQ ID NO: 1. In certain aspects, amino acid changes are minor in nature, that is insertions, substitutions, fragments, deletions, etc. that do not significantly affect the folding and/or activity of the protein (e.g., conservative substitutions; small deletions, typically of one to about 10 amino acids; small amino- or carboxyl-terminal extensions, such as an amino-terminal methionine residue; a small linker peptide; or a small extension that facilitates purification by changing net charge or another function, such as a poly-histidine tag, an antigenic epitope, or a binding domain).

In other embodiments, variants are of such a nature that the physico-chemical properties of the polypeptides are altered, but the activity of eliciting CCK and/or GLP-1 release from, for example, STC-1 cells is not. For example, variants may possess improved solubility, increased substrate specificity, or enhanced biological activity.

With respect to variants of the present invention, the polypeptides are tested using assays described herein to determine whether or not they possess the requisite activity. For example in the case of N-terminal or C-terminal truncations, these polypeptides are tested for CCK and/or GLP-1 activity. Examples of variants and testing using the same are provided in the Examples contained herein.

In certain aspects of the present invention teaching polypeptide variants by substitution, residues are mutated by replacing an amino acid in, for example, SEQ ID NO: 1 with another naturally or non-naturally occurring amino acid. Naturally occurring amino acids include, for example, alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), and valine (V).

In specific aspects of the present invention, the substitutions are conservative substitutions. In other specific aspects, the substitutions are non-conservative substitutions. Conservative and non-conservative amino acid substitutions are known to those of ordinary skill in the art. Generally, a conservative amino acid substitution replaces an amino acid with another amino acid of similar chemical structure, charge, etc. and has no appreciable affect on polypeptide function. Whereas, a non-conservative amino acid substitution replaces an amino acid with another amino acid of dissimilar chemical structure, charge, etc.

To further illustrate, conservative substitutions are, for example, substitutions within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). For example, a basic amino acid can be substituted for another basic amino acid, an acidic amino acid can be substituted for another acidic amino acid, a polar amino acid can be substituted for another polar amino acid, and so forth. Amino acid substitutions that do not generally alter specific activity are known in the art and are described, for example, by H. Neurath and R. L. Hill, 1979, In, The Proteins, Academic Press, New York. Non-conservative substitutions can be thought of as substitutions that are not conservative in nature.

In certain aspects of the present invention teaching polypeptide variants by fragments, deletions, truncations, etc., amino acid residues are removed from SEQ ID NO: 1. Such fragments, deletions, truncations, etc. do not substantially negatively affect the activity of the resulting polypeptide. In specific aspects, truncations from C-terminal, N-terminal, or both C-terminal and N-terminal ends of SEQ ID NO: 1 are a part of the present invention. In other specific aspects, these truncated polypeptides can have as many as five, four, three, two, or one N-terminal amino acids removed without substantially negatively affecting the activity (i.e., CCK, GLP-1, or CCK and GLP-1 eliciting activity). In further other specific aspects, these truncated polypeptides can have as many as five, four, three, two, or one C-terminal amino acids removed without substantially negatively affecting the activity (i.e., CCK, GLP-1, or CCK and GLP-1 eliciting activity). These truncations described herein surprisingly and unexpectedly demonstrate an increase in activity. Exemplary truncated polypeptides of the present invention are provided, for example, as SEQ ID NOs: 2-6 and 8-10 and further detailed in Examples 3 and 4. In specific aspects, truncated polypeptides of SEQ ID NO: 1 are about 500 daltons, about 600 daltons, about 700 daltons, about 800 daltons, about 900 daltons, about 1, kDa, about 1.1 kDa, about 1.2 kDa, about 1.3 kDa, about 1.4 kDa, or about 1.5 kDa in size.

The present invention also relates to isolated polynucleotides encoding polypeptides of the present invention as described herein. It is known and appreciated by one of ordinary skill in the art that a range of different polynucleotides encode a given amino acid sequence as a consequence of the degeneracy of the genetic code. One example of a polynucleotide of the present is SEQ ID NO: 14. As noted above with regard to the degeneracy of the genetic code, polynucleotides of the present invention include equivalent polynucleotides, that is, codon-degeneracy sequences, which are different in sequence, but encode the same polypeptide. These polynucleotide sequences can be determined by one of ordinary skill in the art.

By knowledge of the polypeptides of the present invention as described herein, it is possible to devise a number of partial or full-length polynucleotide sequences such as cDNA and/or genomic clones that encode the range of polypeptides of the present invention. For example, polynucleotides of the present invention may be obtained using degenerate PCR which uses primers designed to target sequences encoding the polypeptides described herein. These primers typically contain multiple degenerate positions. Standard techniques known in the art can be used to determine the range of polynucleotide sequences of the present invention.

The method of polypeptide production does not limit the scope of the present invention. Any methodology of polypeptide production known at the time of the present invention may be employed, as can after-arising methodologies.

For example, peptides of the present invention may be produced by transfection of a host cell with an expression vector comprising the coding sequence for the polypeptide of interest. An expression vector or recombinant plasmid is produced by placing coding sequences for a polypeptide of the present invention in operative association with conventional regulatory control sequences capable of controlling the replication and expression in, and/or secretion from, a host cell. Regulatory sequences include promoter sequences, e.g., CMV promoter, and signal sequences which can be derived from other known proteins. A selected host cell is transfected by conventional techniques with a vector to create a transfected host cell. The transfected cell is then cultured by conventional techniques to produce an engineered polypeptide of the present invention.

Suitable vectors for the cloning and subcloning steps employed in the methods and construction of polypeptides of the present invention may be selected by one of skill in the art. For example, the conventional pUC series of cloning vectors may be used. One vector, pUC19, is commercially available. Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR). Other vector sequences include a poly A signal sequence, such as from bovine growth hormone (BGH) and the betaglobin promoter sequence (betaglopro). The expression vectors useful herein may be synthesized by techniques well known to those skilled in the art.

The components of such vectors, e.g. replicons, selection genes, enhancers, promoters, signal sequences and the like, may be obtained from commercial or natural sources or synthesized by known techniques for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of a polypeptide of the present invention. Host cells useful for the cloning and other manipulations of these cloning vectors are also conventional. However, cells from various strains of E. coli may be used for replication of the cloning vectors and other steps in the construction of polypeptides of the present invention. Suitable host cells or cell lines for expression include mammalian cells such as NSO, Sp2/0, CHO (e.g. DG44), COS, HEK, fibroblast cells (e.g., 3T3), and myeloma cells. Human cells may be used, thus enabling the molecule to be modified with human glycosylation patterns. Alternatively, other eukaryotic cell lines may be employed. The selection of suitable host cells and methods for transformation, culture, amplification, screening and polypeptide production and purification are known in the art.

Bacterial cells may prove useful as host cells suitable for the expression of polypeptides of the present invention (see, e.g., Pluckthun, A., Immunol. Rev., 130:151-188 (1992)). However, due to the tendency of polypeptides expressed in bacterial cells to be in an unfolded or improperly folded form or in a non-glycosylated form, any recombinant polypeptide produced in a bacterial cell would have to be screened for retention of activity. If the molecule expressed by the bacterial cell was produced in a properly folded form, that bacterial cell would be a desirable host, or in alternative aspects the molecule may express in the bacterial host and then be subsequently re-folded. For example, various strains of E. coli used for expression are well-known as host cells in the field of biotechnology. Various strains of B. subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering, 8:277-298, Plenum Press (1986) and references cited therein.

The general methods by which the vectors may be constructed, the transfection methods required to produce the host cells of the invention, and culture methods necessary to produce polypeptides of the present invention from such host cell may all be conventional techniques. Typically, the culture method of the present invention is a serum-free culture method, usually by culturing cells serum-free in suspension. Likewise, once produced, polypeptides of the present invention may be purified from the cell culture contents according to standard procedures of the art, including ammonium sulfate precipitation, affinity columns, column chromatography, gel electrophoresis and the like. Such techniques are within the skill of the art and do not limit this invention. Yet another method of expression of polypeptides of the present invention may utilize expression in a transgenic animal, such as described in U.S. Pat. No. 4,873,316. This relates to an expression system using the animal's casein promoter which when transgenically incorporated into a mammal permits the female to produce the desired recombinant protein in its milk.

Once expressed by the desired method, the polypeptides produced are then examined for activity by use of an appropriate assay as described herein.

Polypeptides of the present invention may also be produced in pure and in large quantities by means of organic synthesis, such as solid phase synthesis. Techniques for polypeptide synthesis are well known. For example, R. B. Merrifield (1963). J. Am. Chem. Soc. 85 (14): 2149-2154, Merrifield (1990) Int. J. Peptide. Protein Res 35: 161-214, Atherton E. et al (1979) Bioorg Chem. 8 351, US2009/0292108 and 2009/0221792 and references therein describe polypeptide synthesis. In addition, the polypeptides may be synthesized using an automatic synthesis machine. Of course, the polypeptides may easily be obtained from a commercial supplier of polypeptides. An exemplary method is described in the following passage. 100 mg Tentagel-S-RAM (Rapp-Polymere) at a load of 0.24 mmol/g is transferred to a commercially available polypeptide synthesizing device (PSMM(Shimadzu)), wherein the polypeptide sequence is constructed step-by-step according to the carbodiimide/HOBt method. The FMOC-amino acid derivatives are pre-activated by adding a 5-fold equimolar excess of di-isopropy-carbodiimide (DIC), diisopropy-ethylamine (DIPEA) and hydroxybenzotriazole (HOBt), and following their transfer into the reaction vessel, mixed with the resin support for 30 minutes. Washing steps are carried out by, for example, additions of DMF and thorough mixing for 1 minute. Cleavage steps are carried out by, for example, the addition of piperidine in DMF and thorough mixing for 4 minutes. Removal of the individual reaction and wash solutions is effected by forcing the solutions through the bottom frit of the reaction vessel. The amino acid derivatives FMOC-Ala, FMOC-Arg(Pbf), FMOC-Asp, FMOC-Gly, FMOC-His(Trt), FMOC-Ile, FMOC-Leu, FMOC-Lys(BOC), FMOC-Pro, FMOC-Ser(tBu) and FMOC-Tyr(tBu) (Orpegen) are employed. When synthesis is completed the resin is dried. The polypeptide amide is subsequently cleaved off by treatment with trifluoracetic acid/TIS/EDT/water (95:2:2:1 vol) for 2 hours at room temperature. By way of filtration, concentration of the solution and precipitation by the addition of ice-cold diethyl ether, the crude product is obtained as a solid. The polypeptide is then purified by RP-HPLC in 0.1% TFA with a gradient of 5 on 60% acetonitrile in 40 minutes at a flow rate of 12 ml/min and evaluation of the elutant by means of a UV detector at 215 nm. The purity of the individual fractions is determined by analytical RP-HPLC and mass spectrometry.

Polypeptides of the present invention may. also be fractionated from a protein hydrolysate composition. Methods of making and fractionating protein hydrolysate compositions are known in the art (see, for example, U.S. Pre-Grant Publication No. 20110257087). For example, polypeptides may be fractionated from a soy protein hydrolysate composition. In other aspects, polypeptides may be fractionated from a protein hydrolysate composition derived from barley, canola, lupin, maize, oat, pea, potato, rice, wheat, animal, egg, or combinations thereof. In alternate aspects, the polypeptides may be fractionated from a combination of different protein hydrolysates. For example, polypeptides may be derived from a soy protein hydrolysate composition combined with a maize protein hydrolysate combination. Alternatively, a soy protein hydrolysate composition may be combined with a canola protein hydrolysate composition and a wheat protein hydrolysate composition. In still other aspects, polypeptides may be fractionated from a protein hydrolysate composition derived from a combination of soy and at least one other protein source selected from the group consisting of barley, canola, lupin, maize, oat, pea, potato, rice, wheat, animal, dairy, and egg.

### II. METHODS OF INDUCING CCK AND/OR GLP-1 AND METHODS OF INDUCING SATIETY

In certain aspects, the present invention provides a method of eliciting release of CCK and/or GLP-1 from a cell comprising contacting said cell with an effective amount of a polypeptide described herein. The polypeptide provided can be in any acceptable form (e.g., as a part of an ingredient in a food product, a supplement, etc.). Determining the release of CCK and GLP-1 is known in the art. For example, an appropriate sample (e.g., cell media, serum, and so forth) can be measured before and after being subjected to a polypeptide of invention to determine whether there is an increase in CCK and/or GLP-1. These measurements are done, for example, using commercially available immunoassays.

In other certain aspects, the present invention provides a method of inducing satiety comprising providing an effective amount of a polypeptide described herein. The polypeptide provided can be in any acceptable form (e.g., as a part of an ingredient in a food product, a supplement, etc.). Determining whether satiety is induced is known in the art. For example, biomarkers (e.g., CCK and GLP-1) can be used as a means of measuring satiety, as can more subjective measures including visual analogue scales, which are often composed of lines (of varying length) with words anchored at each end, describing the extremes (e.g., “I have never been more hungry”-“I am not hungry at all”).

### III. PRODUCTS COMPRISING A POLYPEPTIDE OF THE PRESENT INVENTION

Certain aspects of the present invention encompass a food product comprising an edible material and a polypeptide described herein. The selection of a particular polypeptide to combine with an edible material can and will vary depending upon the desired food product.

The selection of the appropriate edible material will vary depending on the desired food product The edible material may be a plant-derived material (e.g., a vegetable juice, a cereal product, etc.), an animal-derived material (e.g., a dairy product, an egg product, etc.), or a biomaterial (e.g., a protein, a carbohydrate, a lipid, etc.) isolated from a plant-derived material or an animal-derived material, and so forth.

Certain food products of the present invention include, for example, hot or cold cereals, bars, baked goods, beverages, yogurts, desserts, snacks, pastas, and meats (including poultry and seafood).

In one aspect of the present invention, the food product is a liquid or dry blended beverage. Non-limiting examples of liquid or dry blended beverages include fruit juices, fruit drinks, fruit-flavored drinks, vegetable drinks, nutritional drinks, energy drinks, sports drinks, soy milk drinks, flavored soy drinks, rice milk-based drinks, flavored milk drinks, yogurt-based drinks, infant formula, tea-based beverages, coffee-based beverages, meal replacement drinks, protein shakes, supplement beverages, weight management beverages, combinations and dried blended beverages thereof.

The edible material comprising the liquid or dried blended beverage can and will vary. Non-limiting examples of suitable edible materials include fruit juices, vegetable juices, skim milk, reduced fat milk, 2% milk, whole milk, cream, evaporated milk, yogurt, buttermilk, chocolate, cocoa powder, coffee, tea, and so forth.

The beverage product may further comprise natural and artificial sweetening agents (e.g., glucose, sucrose, fructose, maltodextrin, sucralose, aspartame, saccharin, stevia, corn syrup, honey, maple syrup, etc.), flavoring agents (e.g., chocolate, cocoa, chocolate flavor, vanilla extract, vanilla flavor, fruit flavors, etc.), emulsifying or thickening agents (e.g., lecithin, carrageenan, cellulose gum, cellulose gel, starch, gum arabic, xanthan gum, etc.), stabilizing agents, lipid materials (e.g., canola oil, sunflower oil, high oleic sunflower oil, fat powder, etc.), preservatives and antioxidants (e.g., potassium sorbate, sorbic acid, BHA, BHT, TBHQ, rosemary extract, vitamins A, C and E and derivatives thereof, and various plant extracts such as those containing carotenoids, tocopherols or flavonoids having antioxidant properties, etc.), coloring agents, vitamins, minerals, and combinations thereof.

In certain aspects of the present invention, the food product is a food bar, such as a granola bar, a cereal bar, a nutrition bar, a meal replacement bar, or an energy bar.

In other certain aspects of the present invention, the food product is a cereal-based product. Non-limiting examples of cereal-based food products include breakfast cereals, breakfast bars, pasta, breads, baked products (e.g., cakes, pies, rolls, cookies, crackers), and snack products (e.g., chips, pretzels, etc.). The edible material of a cereal-based food product may be, for example, derived from wheat (e.g., bleached flour, whole wheat flour, wheat germ, wheat bran, etc.), corn (e.g., corn flour, cornmeal, cornstarch, etc.), oats (e.g., puffed oats, oatmeal, oat flour, etc.), rice (e.g., puffed rice, rice flour, rice starch), and so forth.

In another aspect of the present invention, the food product is a “solid” dairy-based product. Non-limiting examples of suitable “solid” dairy-based food products include a hard cheese product, soft cheese product, ice cream product, yogurt product, frozen yogurt product, whipped dairy-like product, sherbet, etc.

In further aspect of the present invention, the food product is a meat product or a meat analog product. Examples of meat food products include, for example, processed meats, comminuted meats, and whole muscle meat products. The meat material may be animal meat or seafood meat. The meat analog may be a textured vegetable or dairy protein that mimics animal or seafood meat in texture. The meat analog may be part or all of the meat material in a meat food product.

In another aspect of the present invention, a product containing a polypeptide described herein is a supplement.

### IV. DEFINITIONS

Unless otherwise noted, technical terms are used according to conventional usage. Definitions of common scientific technical terms may be found, for example, in McGraw-Hill Dictionary of Scientific & Technical Terms published by McGraw-Hill Healthcare Management Group; Benjamin Lewin, Genes VIII, published by Oxford University Press; Kendrew et al. (eds.), The Encyclopedia of Molecular Biology, published by Blackwell Publishers; and Robert A. Meyers (ed.), Molecular Biology and Biotechnology: a Comprehensive Desk Reference, published by Wiley, John & Sons, Inc; and other similar technical references.

As used herein, “a” or “an” may mean one or more. As used herein in conjunction with the word “comprising”, the words “a” or “an” may mean one or more than one. As used herein “another” may mean at least a second or more. Furthermore, unless otherwise required by context, singular terms shall include pluralities and plural terms shall include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, “comprising” and all its forms and tenses (including, for example, comprise and comprised) is synonymous with “including,” “containing,” or “characterized by,” and is inclusive or open-ended language and does not exclude any additional, unrecited element, step, or ingredient. As used herein, “consisting” and all its forms and tenses (including, for example, consist and consisted) is closed language and excludes any element, step, or ingredient not specified. As used herein, “consisting essentially of” and all its forms and tenses limits the scope of the invention to the specified element, step, or ingredient and those that do not materially affect the basic and novel characteristic(s) of the claimed invention. Applicants note that certain embodiments recite the transitional phrase “comprising.” Wherever Applicants have recited this transitional phrase, the transitional phrase consisting or consisting essentially of have also been contemplated by Applicants and are part of the invention.

As used herein, “effective amount” means an amount of a polypeptide of the invention that when contacted with a cell (e.g., an STC-1 cell) is able to elicit from said cell CCK release, GLP-1 release, or combinations thereof. For example, an effective amount is illustrated as the quantities and concentrations of SEQ ID NO: 1 and variants thereof used in the Examples, which one of ordinary skill in the art could adjust based on the circumstances (e.g., cells in isolation compared to highly complex living multicellular organisms).

As used herein, “food product” means a product containing proteins, carbohydrates, and/or fats and is intended for consumption and to be used as a source of sustenance or nourishment (e.g., growth, repair, and vital processes and to furnish energy). For example, a food product includes, without being limited to, hot or cold cereals, bars (e.g., a granola bar, a cereal bar, a nutrition bar, a meal replacement bar, or an energy bar), baked goods, beverages (e.g., liquid and dry blended), yogurts, desserts, snacks, pastas, meats, including poultry and seafood (e.g., processed meats, comminuted meats, and whole muscle meats), cereal-based products (e.g., breakfast cereals, breakfast bars, pasta, breads, baked products (e.g., cakes, pies, rolls, cookies, crackers), and snack products (e.g., chips, pretzels, etc.)), and dairy-based products (e.g., hard cheeses, soft cheeses, ice cream, yogurt, frozen yogurt, whipped dairy-like products, sherbet, etc.).

As used herein, “isolated” or “purified” means a polypeptide or polynucleotide that is removed from at least one component with which it is naturally associated. For example, a polypeptide may be at least 1% pure, e.g., at least 5% pure, at least 10% pure, at least 20% pure, at least 40% pure, at least 60% pure, at least 80% pure, at least 90% pure, or at least 95% pure, as determined by SDS-PAGE, and a polynucleotide may be at least 1% pure, e.g., at least 5% pure, at least 10% pure, at least 20% pure, at least 40% pure, at least 60% pure, at least 80% pure, at least 90% pure, or at least 95% pure, as determined by agarose electrophoresis.

As used herein, “supplement” means a composition intended to supplement the diet by providing specific nutrients or other molecules, as opposed to bulk calories. For example, a supplement can be a solid, semi-solid, or liquid form (e.g., tableted or encapsulated) of providing a polypeptide of the invention. In addition to a polypeptide of the invention, a supplement may contain any one or more of the following items: a vitamin, a mineral, an herb, an amino acid, an essential fatty acid, and other substances traditionally or otherwise that may be thought of or used in a supplement.

As used, herein, “variant” means a sequence that is different compared to a sequence provided as a SEQ ID NO: 1, but nonetheless contains part of SEQ ID NO: 1. Variants related to polypeptides of the invention are also able to elicit CCK and/or GLP-1 release from, for example, STC-1 cells. A variant of the invention includes, without limitation, insertions, substitutions, fragments, deletions, C-terminal truncations, and N-terminal truncations of SEQ ID NO: 1.

### V. EXAMLES

### Example 1

### CCK Activity

SEQ ID NO: 1 was chemically synthesized. To assay for CCK release activity, SEQ ID NO: 1 was dissolved in Dulbecco's phosphate buffered saline (D-PBS) at 10 mg/ml. This solution was then diluted to between 0.002 and 4 mg/ml with d-PBS, then added to media of STC-1 cells (passage 25 to 28) in 1:1 ratio of D-PBS and Dulbecco's Modified Eagle's Medium (DMEM-high glucose) and incubated with the cells at 37° C. (99° F.) and a CO₂ level of 5% for 4 hours. The solution containing SEQ ID NO: 1 was added to cells at a variety of concentrations ranging from 0.001 to 2 mg/ml to generate a dose-response curve.

Following the incubation period, concentrations of CCK released by STC-1 cells into the media were assayed using a commercially available immunoassay kit for CCK detection from Phoenix Pharmaceuticals, Burlingame, Calif. (catalogue number EK-069-04). Assays were performed according to the manufacturer's instructions using a standard curve covering a range of concentrations from 0.4 to 1000 pg/well. Absorbance was measured at a wavelength of 450 nm.

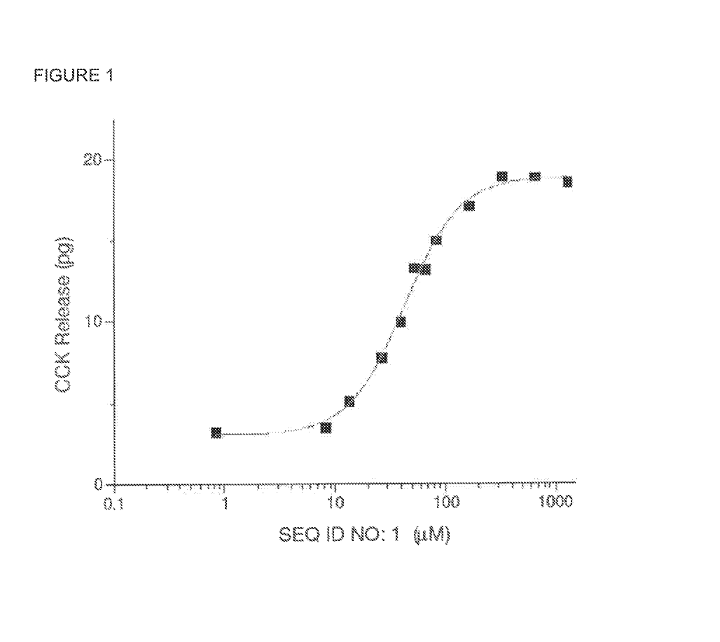

FIG. 1 depicts the CCK release dose-response for SEQ ID NO: 1. CCK quantities obtained from each tested concentration of SEQ ID NO: 1 were plotted vs. log concentration of peptide. A dose-response curve was then generated by fitting the data with a logistic function using OriginLab graphing software. The EC₅₀ was extrapolated from the fit data and found to be 43 μM.

### Example 2

### GLP-1 Activity

SEQ ID NO: 1 was chemically synthesized. To assay for GLP-1 release activity, SEQ ID NO: 1 was dissolved in Dulbecco's phosphate buffered saline (D-PBS) at 10 mg/ml. This solution was diluted to between 0.002 and 4 mg/ml with D-PBS, then added to media of STC-1 cells (passage 25 to 28) in 1:1 ratio of D-PBS and Dulbecco's Modified Eagle's Medium (DMEM-high glucose) and incubated with the cells at 37° C. (99° F.) and a CO₂ level of 5% for 2 hours. The solution containing SEQ ID NO: 1 was added to cells at a variety of concentrations ranging from 0.001 to 2 mg/ml to generate a dose-response curve.

Following the incubation period, concentrations of GLP-1 released by the STC-1 cells into the media were assayed using a commercially available immunoassay kit for GLP-1 detection from Phoenix Pharmaceuticals, Burlingame, CA (catalogue number EK-028-11). Assays were performed according to the manufacturer's instructions using a standard curve covering a range of concentrations from 0.4 to 1000 pg/well. Absorbance was measured at a wavelength of 450 nm.

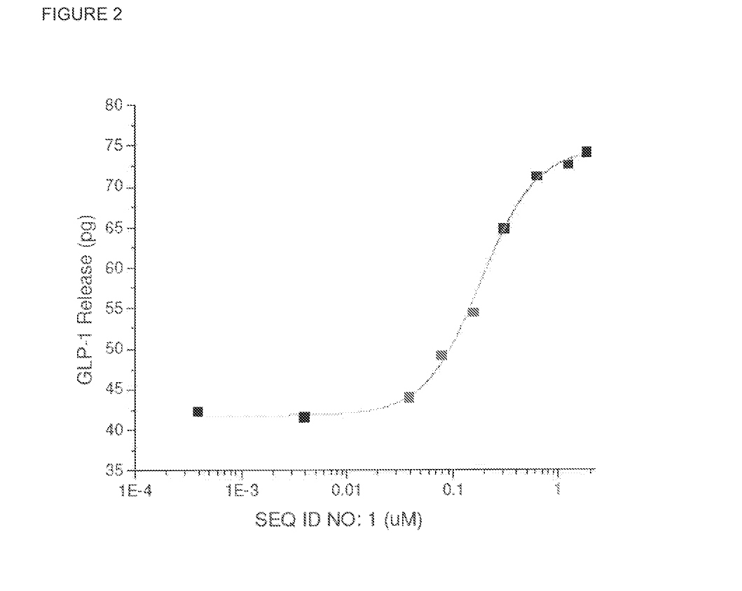

FIG. 2 depicts GLP-1 dose-response curve for SEQ ID No: 1. GLP-1 quantities obtained from each tested concentration of SEQ ID NO: 1 were plotted vs. log concentration of peptide. A dose-response curve was then generated by fitting the data with a logistic function using OriginLab graphing software. The EC₅₀ was extrapolated from the fit data and found to be 186 μM.

### Example 3

### Sequence Variants Demonstrate CCK Activity

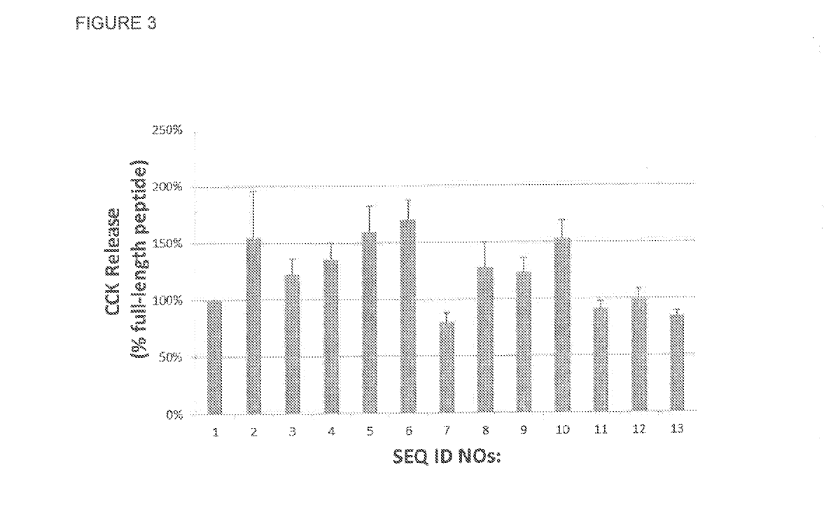

C- and N-terminal truncation variants of SEQ ID NO: 1 were chemically synthesized and assayed for CCK release activity. Each variant was assayed for CCK activity at 2 mg/ml using the method described in Example 1. The results of these assays are shown in FIG. 3. The peptide variants are described in Table 1.

TABLE 1

| SEQ ID Nos | Amino Acid Sequence |
|---|---|
| 1 | NH2-TSLDFPALSWLRL-COOH |
| 2 | NH2-SLDFPALSWLRL-COOH |
| 3 | NH2-LDFPALSWLRL-COOH |
| 4 | NH2-DFPALSWLRL-COOH |
| 5 | NH2-FPALSWLRL-COOH |
| 6 | NH2-PALSWLRL-COOH |
| 7 | NH2-ALSWLRL-COOH |
| 8 | NH2-TSLDFPALSWLR-COOH |
| 9 | NH2-TSLDFPALSWL-COOH |
| 10 | NH2-TSLDFPALSW-COOH |
| 11 | NH2-TSLDFPALS-COOH |
| 12 | NH2-TSLDFPAL-COOH |

In each case the peptide variants demonstrated varying amounts of CCK releasing activity. Serial truncations of the first five N-terminal amino acids demonstrated activities significantly greater than the full-length peptide, as did serial truncations of the first three C-terminal amino acids (see FIG. 3).

### Example 4

### Sequence Variants Demonstrate GLP-1 Activity

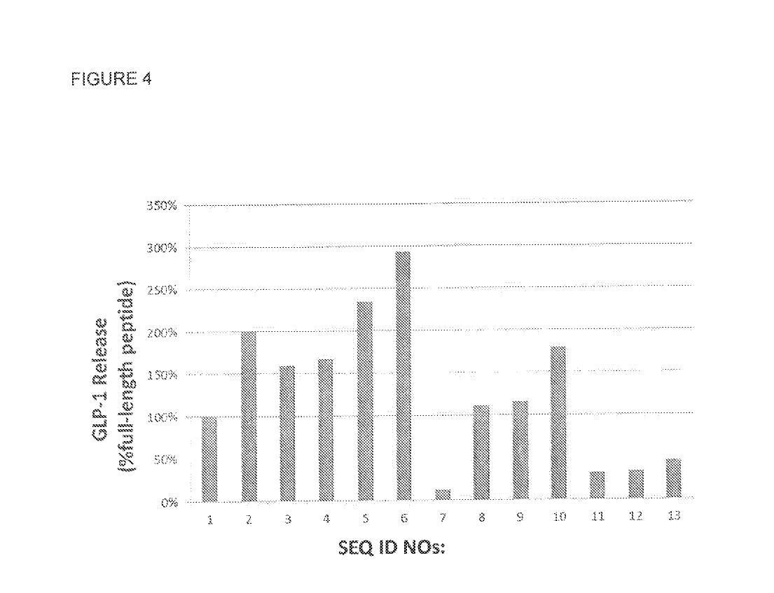

C- and N-terminal truncation variants of SEQ ID NO: 1 were chemically synthesized and assayed for GLP-1 release activity. Each variant was assayed for GLP-1 activity at 2 mg/ml using the method described in Example 2. The results of these assays are shown in FIG. 4. The peptide variants are described in Table 1.

In each case the peptide variants demonstrated varying amounts of GLP-1 releasing activity. Serial truncations of the first five N-terminal amino acids demonstrated activities significantly greater than the full-length peptide, as did serial truncations of the first three C-terminal amino acids (see FIG. 4).

### REFERENCES

All patents and publications mentioned and/or cited herein are incorporated by reference to the same extent as if each individual publication was specifically and individually indicated as having been incorporated by reference in its entirety.

SEQUENCES

SEQ ID NO: 1-TSLDFPALSWLRL
SEQ ID NO: 2-SLDFPALSWLRL
SEQ ID NO: 3-LDFPALSWLRL
SEQ ID NO: 4-DFPALSWLRL
SEQ ID NO: 5-FPALSWLRL
SEQ ID NO: 6-PALSWLRL
SEQ ID NO: 7-ALSWLRL
SEQ ID NO: 8-TSLDFPALSWLR
SEQ ID NO: 9-TSLDFPALSWL
SEQ ID NO: 10-TSLDFPALSW
SEQ ID NO: 11-TSLDFPALS
SEQ ID NO: 12-TSLDFPAL
SEQ ID NO: 13-TSLDFPA
SEQ ID NO: 14-ACT TCT TTA GAT TTT
CCT GCT CTT TCT TGG TTA AGA TTA

1. An isolated polypeptide comprising the amino acid sequence of SEQ ID NO: 1 or variant thereof.

2. The polypeptide of claim 1, wherein said variant is at least about 50% identical to SEQ ID NO: 1 and is able to elicit cholecystokinin (CCK) release, glucagon-like polypeptide-1 (GLP-1) release, or combinations thereof, from STC-1 cells when said polypeptide is contacted with said cells in an effective amount.

3. The polypeptide of claim 2, wherein said variant is at least about 75% identical to SEQ ID NO: 1 and is able to elicit CCK release, GLP-1 release, or combinations thereof, from STC-1 cells when said polypeptide is contacted with said cells in an effective amount.

4. The polypeptide of claim 3, wherein said variant is at least about 90% identical to SEQ ID NO: 1 and is able to elicit CCK release, GLP-1 release, or combinations thereof, from STC-1 cells when said polypeptide is contacted with said cells in an effective amount.

5. The polypeptide of claim 2, wherein said variant is an N-terminal truncation, C-terminal truncation, or combination thereof.

6. A product comprising
(a) an edible material; and
(b) an isolated polypeptide comprising the amino acid sequence of SEQ ID NO: 1 or variant thereof.

7. The product of claim 6, wherein said product is a food product.

8. The product of claim 6, wherein said product is a supplement.

9. The product of claim 6, wherein said variant is at least about 50% identical to SEQ ID NO: 1 and is able to elicit CCK release, GLP-1 release, or combinations thereof, from STC-1 cells when said polypeptide is contacted with said cells in an effective amount.

10. The product of claim 9, wherein said variant is at least about 75% identical to SEQ ID NO: 1 and is able to elicit CCK release, GLP-1 release, or combinations thereof, from STC-1 cells when said polypeptide is contacted with said cells in an effective amount.

11. The product of claim 10, wherein said variant is at least about 90% identical to SEQ ID NO: 1 and is able to elicit CCK release, GLP-1 release, or combinations thereof, from STC-1 cells when said polypeptide is contacted with said cells in an effective amount.

12. The polypeptide of claim 6, wherein said variant is an N-terminal truncation, C-terminal truncation, or combination thereof.

13. A method of eliciting CCK release, GLP-1 release, or combinations thereof, from a cell comprising contacting said cell with a polypeptide comprising the amino acid sequence of SEQ ID NO: 1 or variant thereof.

14. The method of claim 13, wherein said variant is at least about 50% identical to SEQ ID NO: 1 and is able to elicit CCK release, GLP-1 release, or combinations thereof, from STC-1 cells when said polypeptide is contacted with said cells in an effective amount.

15. The method of claim 14, wherein said variant is at least about 75% identical to SEQ ID NO: 1 and is able to elicit CCK release, GLP-1 release, or combinations thereof, from STC-1 cells when said polypeptide is contacted with said cells in an effective amount.

16. The method of claim 15, wherein said variant is at least about 90% identical to SEQ ID NO: 1 and is able to elicit CCK release, GLP-1 release, or combinations thereof, from STC-1 cells when said polypeptide is contacted with said cells in an effective amount.

17. The polypeptide of claim 13, wherein said variant is an N-terminal truncation, C-terminal truncation, or combination thereof.
